(19) United States
(12) Patent Application Publication
(10) Pub. No.: US 2025/0147040 A1
(43) Pub. Date: May 8, 2025

(54) SURFACE-INDUCED DISSOCIATION DEVICES AND METHODS

(71) Applicant: Ohio State Innovation Foundation, Columbus, OH (US)

(72) Inventors: Sean CLEARY, Columbus, OH (US); Chen DU, White Plains, NY (US); Vicki WYSOCKI, Worthington, OH (US)

(21) Appl. No.: 18/835,636

(22) PCT Filed: Feb. 3, 2023
(86) PCT No.: PCT/US2023/061983

§ 371 Date: Aug. 2, 2024

Related U.S. Application Data
(60) Provisional application No. 63/306,920, filed on Feb. 4, 2022.

(51) Int. Cl. G01N 33/68; H01J 49/00

(52) U.S. Cl. CPC G01N 33/6848; H01J 49/0068; G01N 2333/005

(57) ABSTRACT

Systems and methods for surface-induced dissociation (SID) are disclosed herein. According to one embodiment of the present disclosure, a system for characterizing the structure of a sample includes an SID device configured to receive the sample, the sample including a plurality of viral capsids; a charge detection mass spectrometer (CDMS) operably coupled to the surface induced dissociation device and configured to receive fragmented viral capsids from the surface induced dissociation device, where the CDMS is configured to determine a characterization of the structure of the viral capsids.

### CROSS-REFERENCE TO RELATED APPLICATIONS

This Application claims priority to, and benefit under 35 U.S.C. § 119(e) of, U.S. Provisional Patent Application No. 63/306,920 filed Feb. 4, 2022, which is hereby incorporated by reference herein in its entirety.

### STATEMENT REGARDING GOVERNMENT SUPPORT

This invention was made with government support under GM128577 awarded by National Institutes of Health. The government has certain rights in the invention.

### BACKGROUND

Mass spectrometry has recently become a powerful tool for structural biology. The observation that the tertiary and quaternary structure of proteins and their complexes can be kinetically trapped after transfer from solution to the gas phase through a combination of careful sample preparation (exchanging into a volatile buffer, e.g. ammonium acetate) and soft electrospray ionization led to the eventual coining of the term “native mass spectrometry” (nMS). This was followed by the emergence of improved mass spectrometric technologies (e.g. ion mobility, surface-induced dissociation, ultraviolet photodissociation, Q-IM-TOFs, Fourier Transform Ion Cyclotron Resonance (FT-ICR), and high mass Orbitraps for studying high-mass species.

Surface-induced dissociation (SID), in which the collision target is a surface (usually coated with a fluorocarbon self-assembled monolayer but other coatings or untreated metal are acceptable for some precursors) rather than a small gas molecule, has been shown to generate more symmetrically charged fragments or proportionally charged fragments that are reminiscent of the three-dimensional structure of protein complexes, in part because SID consists of a single (or low number) collision with higher energy conversion versus many low energy collisions in CID. SID can provide a wealth of information, including overall conformational changes, subunit interconnectivity and interaction strength (which can be used to constrain computer models), and subunit-ligand interactions.

It is with respect to these and other considerations that the various embodiments described below are presented.

### SUMMARY

In some aspects, the present disclosure relates to devices and methods for surface-induced dissociation (SID).

In one aspect, the present disclosure relates to a system for characterizing the structure of a sample which, in one embodiment, includes a surface induced dissociation (SID) device configured to receive the sample, the sample including a plurality of viral capsids; a charge detection mass spectrometer (CDMS), operably coupled to the surface induced dissociation device and configured to receive viral capsids from the surface induced dissociation device, where the CDMS is configured to determine a characterization of the structure of adeno-associated viral vector capsids.

In some embodiments, the CDMS includes a nano-electrospray ionization source.

In some embodiments, the SID device is configured to perform SID on the viral capsids.

In some embodiments, the SID device is configured to perform SID on an impurity in a sample.

In some embodiments, the SID voltage is less than 100V.

In some embodiments, the SID voltage is greater than 230 volts.

In some embodiments, the characterization of the plurality of viral capsids includes an intact mass measurement of the viral capsids.

In some embodiments, the characterization of the plurality of viral capsids includes the respective ratios of empty viral capsids, partially-filled viral capsids, and fully filled viral capsids.

In another aspect, the present disclosure relates to a method of characterizing the structure of viral capsids which, in one embodiment, includes: inputting a plurality of viral capsids into an SID device; fragmenting the plurality of viral capsids using the SID device to create a plurality of adeno-associated viral vector fragments; inputting the plurality of viral capsid fragments into a charge detection mass spectrometer; and determining a characterization of the plurality of viral capsids using the CDMS.

In some embodiments, the CDMS includes a nano-electrospray ionization source.

In some embodiments, fragmenting the plurality of viral capsids using the surface induced dissociation device includes configuring the surface induced dissociation device to produce a collision voltage of greater than 230 volts.

In some embodiments, the method further includes outputting a plurality of peaks.

In some embodiments, the plurality of peaks correspond to monomer, dimer, trimer, pentamer, 9mer, 15mer, 24mer, 30mer, 36, 45mer, and 57mer fragments.

In some embodiments, the characterization of the plurality of viral capsids includes the respective ratios of a number of empty viral capsids, a number of partially-filled viral capsids, and a number of fully filled viral capsids.

In yet another aspect, the present disclosure relates to a method of characterizing the structure of sample which, in one embodiment, includes: inputting the sample ions into an SID device, the sample comprising a plurality of viral capsids and an impurity, where the SID device is configured to fragment the impurity without fragmenting the viral capsids; fragmenting the impurity using the SID device; inputting the plurality of viral capsids into a CDMS; and determining a characterization of the plurality of viral capsids using the charge detection mass spectrometer.

In some embodiments, the sample is prepared using a nano-electrospray ionization source.

In some embodiments, the impurity includes a salt.

In some embodiments, the impurity includes a solvent ion.

In some embodiments, the impurity includes a byproduct of an electrospray process.

In some embodiments, the SID device is configured to produce an SID voltage less than 100V.

In some embodiments, the characterization of the plurality of viral capsids includes an intact mass measurement of the viral capsids.

In some embodiments, the characterization of the plurality of adeno-associated viral vectors includes the respective ratios of empty viral capsids, partially-filled viral capsids, and fully filled viral capsids.

Other aspects and features according to the example embodiments of the present disclosure will become apparent to those of ordinary skill in the art, upon reviewing the following detailed description in conjunction with the accompanying figures.

### BRIEF DESCRIPTION OF THE FIGURES

The accompanying drawings, which are incorporated in and constitute a part of this specification, illustrate embodiments and together with the description, serve to explain the principles of the disclosed technology.

FIG. 1 illustrates a system configured to perform surface induced dissociation (SID) and charge spectrum mass spectrometry (CDMS), in accordance with an embodiment of the present disclosure.

FIG. 2A illustrates a method of characterizing the structure of viral capsids by using SID and CDMS to fragment viral capsids.

FIG. 2B illustrates a method of characterizing the structure of AAV vectors by using SID and CDMS, and without fragmenting AAV's.

FIG. 3A illustrates a mass-m/z plot of an SID spectra from empty AAV8 capsid acquired with an SID voltage of 125V, according to an example embodiment of the present disclosure.

FIG. 3B illustrates a mass-m/z plot of an SID spectra from empty AAV8 capsid acquired with an SID voltage of 235V, according to an example embodiment of the present disclosure.

FIC. 3C illustrates the low-mass region of FIG. 3B.

FIG. 3D illustrates a SID-CDMS spectrum processed and deconvolved by UniDec CD, where the SID voltage was 125V.

FIG. 3E illustrates an example spectra from the low-mass region of FIG. 3D.

FIG. 3F illustrates a SID-CDMS spectrum processed and deconvolved by UniDec CD, where the SID voltage was 235V.

FIG. 3G illustrates interface areas of AAV8 60mer as determined by PISA analysis (PDB ID: 2QA0). Trimer has a higher interface area, consistent with the trimeric fragments observed in SID.

FIG. 4 illustrates an example computing device.

### DETAILED DESCRIPTION

In some aspects, the present disclosure relates to surface-induced dissociation (SID) devices and methods. Although example embodiments of the present disclosure are explained in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an” and “the” include plural referents unless the context clearly dictates otherwise. Certain values may be expressed in terms of ranges “from” one value “to” another value. When a range is expressed in terms of “from” a particular lower value “to” a particular higher value, or “from” a particular higher value “to” a particular lower value, the range includes the particular lower value and the particular higher value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “(n)” corresponds to the n^{th} reference in the list. For example, (3) refers to the 3^{rd} reference in the list, namely Dobnik D, Kogovsek P, Jakomin T, Kosir N, Tusek Znidaric M, Leskovec M, et al. Accurate quantification and characterization of adeno-associated viral vectors. Front Microbiol. 2019; 10:1570. All references cited and discussed in this specification are incorporated herein by reference in their entireties and to the same extent as if each reference was individually incorporated by reference.

A detailed description of aspects of the present disclosure, in accordance with various example embodiments, will now be provided with reference to the accompanying drawings. The drawings form a part hereof and show, by way of illustration, specific embodiments and examples. In referring to the drawings, like numerals represent like elements throughout the several figures. Some experimental data are presented herein for purposes of illustration and should not be construed as limiting the scope of the present disclosure in any way or excluding any alternative or additional embodiments.

Precursor ions that impact the surface of an SID device may fragment and become product ions. Throughout the present disclosure, “product ions” may alternatively be referred to as “products,” “fragments,” or “fragment ions.” Similarly, ions that interact with the surface may be referred to as “activated” or “activated ions.” Precursor ions may also be referred to as “precursors” or “precursor complexes.”

The present disclosure includes devices for SID. SID devices can include a collision surface, a guide configured to guide precursor ions from a pre-SID region to the collision surface (e.g., a deflector or deflecting voltage), and an extractor (or other device) configured to extract ions off the collision surface after collision with the collision surface. For example, in some embodiments, the device also includes a radiofrequency (RF) device configured to collect and/or transmit the extracted ions.

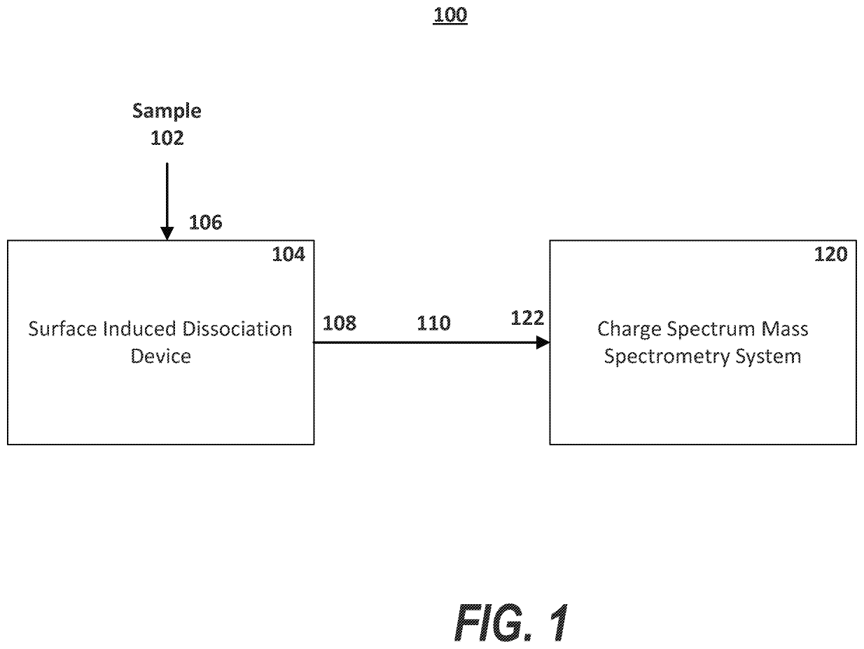

With reference to FIG. 1, embodiments of the present disclosure include systems for characterizing the structure of a sample. The example system 100 shown in FIG. 1 can include an SID device 104. The SID device 104 can include an input 106 configured to receive a sample, and an output 108 configured to output SID products 110.

The example system 100 can further include a charge spectrum mass spectrometer (CDMS) 120 with an input 122. The input 122 can be configured to receive the SID products 110 from the SID device 104. It should be understood that the CDMS 120 can be replaced with other spectrometers in other embodiments of the present disclosure, and that the CDMS described herein is a non-limiting example. Alternatively or additionally, it should be understood that in some embodiments the spectrometer (e.g., a CDMS) can include an Orbitrap.

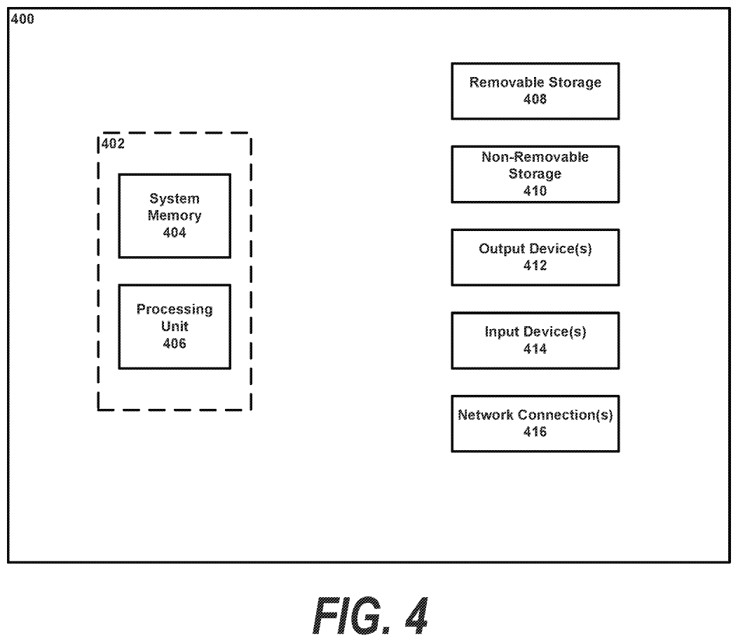

The CDMS can optionally include a computing device (e.g., the computing device 400 illustrated in FIG. 4) that can be configured to evaluate the products and output the results of an evaluation of the products. Optionally, in embodiments that are configured to evaluate viral capsids, the results can include a characterization of viral capsids. A non-limiting example characterization of the viral capsids is the intact mass measurement of the viral capsids. Alternatively or additionally, the characterization of the viral capsids can be the respective ratios of empty viral capsids, partially-filled viral capsids, and fully filled viral capsids.

In some embodiments, the sample input to the SID device 104 includes viral capsids. The viral capsids can optionally be prepared by electrospray ionization. The SID device 104 can optionally be configured so that it fragments the viral capsids, or so that it does not fragment the viral capsids, or reduces the number of viral capsids fragmented. As a non-limiting example, in some embodiments, an SID voltage of less than 100V avoids fragmenting most or all of the viral capsids. As another non-limiting example, in some embodiments, an SID voltage of greater than 235V can result in the fragmentation of empty viral capsids. It should be understood that these voltages and ranges of voltages are intended only as non-limiting examples, and that in other embodiments different voltages can be used to fragment or avoid fragmentation of different proportions of viral capsids. For example, in some embodiments, the voltage used can be between 100V and 235V.

Optionally, in some embodiments, the SID voltage can be selected to fragment molecules in the sample 102 other than viral capsids. For example, in embodiments where a nano-electrospray process is used to prepare the sample 102, the electrospray process can introduce impurities, for example salt and/or solvent adducted ions that can be fragmented at lower SID voltages that are insufficient to fragment the viral capsids. Fragmenting salt and/or solvent adducted ions can be beneficial to separating them from the viral capsids in the CDMS measurements (e.g., spectra).

Additionally, it should be understood that the AAV's described herein are only non-limiting examples of viral capsids. For example, the present disclosure contemplates that any viral capsid used for Gene Therapy can be used. In some embodiments of the present disclosure, the AAV's can be replaced with any other viral vector (e.g., viral vectors for gene therapy). Additional non-limiting examples of viral vectors that can be used include adenovirus (Ad), herpes simplex virus (HSV), retrovirus and lentivirus.

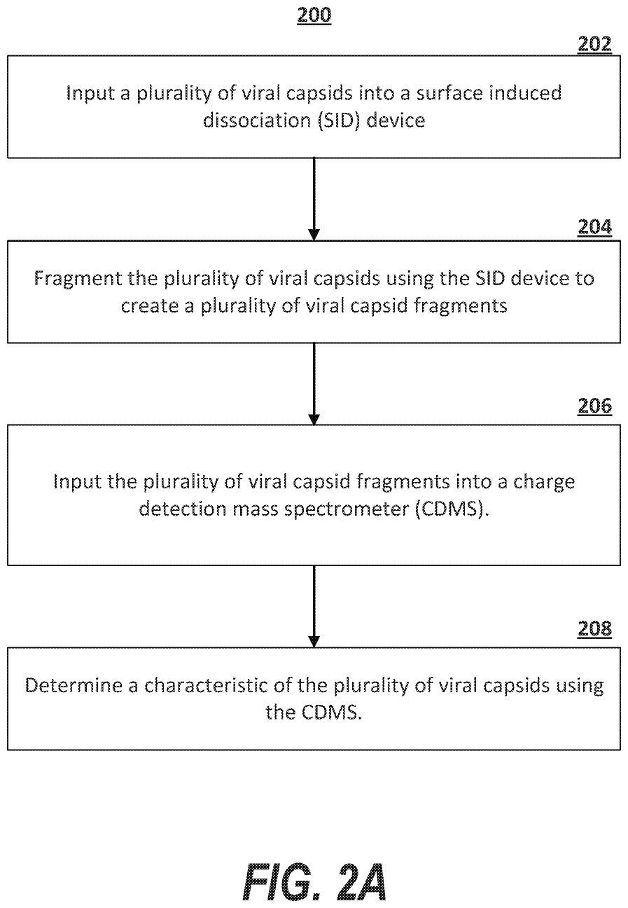

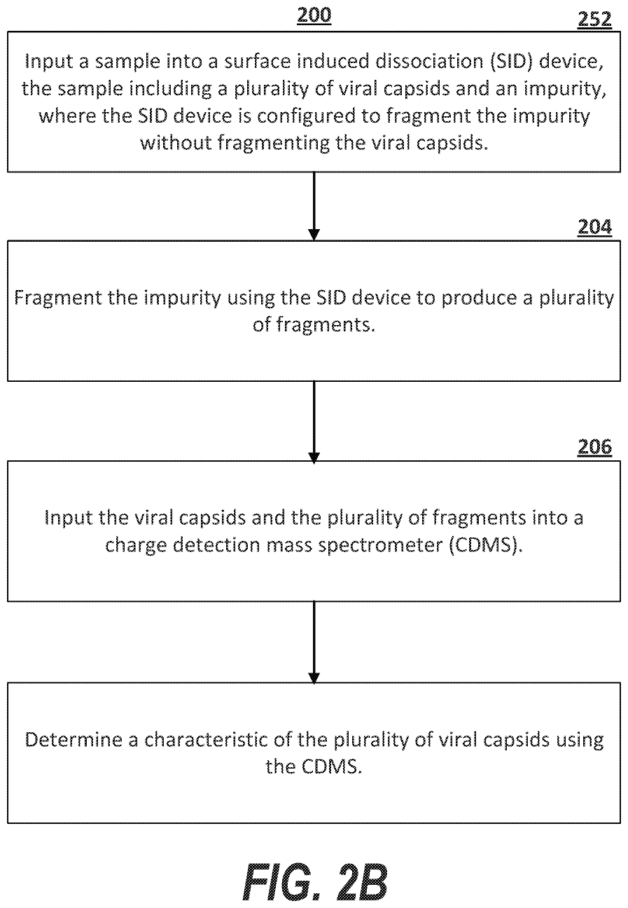

With reference to FIGS. 2A-2B, embodiments of the present disclosure include methods of performing SID on viral capsids. Optionally, the methods illustrated in FIGS. 2A-2B can be performed using the system 100 illustrated in FIG. 1.

With reference to FIG. 2A, an example method 200 of characterizing the structure of viral vectors is shown. At step 202, the method 200 can include inputting viral capsids into a surface induced dissociation device. As described with reference to FIG. 1, the viral capsids can optionally be prepared using nano-electrospray process or using a nano-electrospray source.

At step 204, the method 200 can include fragmenting the plurality of viral capsids using the surface induced dissociation device to create a plurality of viral capsid fragments. Optionally, the surface induced dissociation device can be configured to produce a collision voltage of greater than 230 volts.

At step 206, the method can include inputting the plurality of viral capsid fragments into a charge detection mass spectrometer.

At step 208, the method can include determining a characterization of the plurality of viral capsids using the charge detection mass spectrometer. The charge detection mass spectrometer can output information characterizing the viral capsids. As a non-limiting example, the information characterizing the viral capsids can include graphs including peaks representing the masses of the fragments. As another non-limiting example, the peaks can correspond to monomer, dimer, trimer, pentamer, 9mer, 15mer, 24mer, 30mer, 36, 45mer, and 57mer fragments. It should be understood that the peaks can optionally correspond to multiples of 3 (e.g., 9mer, 15mer, 24mer, 30mer, 36mer, 45mer and 57mer are all multiples of 3). As yet another non-limiting example, the information characterizing the viral capsids can include respective ratios of empty viral capsids, partially-filled viral capsids, and fully filled viral capsids.

With reference to FIG. 2B, embodiments of the present disclosure can include methods of characterizing viral capsids without fragmenting the viral capsids. An example method 250 of characterizing viral capsids without fragmenting the viral capsids is shown in FIG. 2B.

At step 252, the method 250 can include inputting a sample including viral capsids into a surface induced dissociation device. As a non-limiting example, the viral capsids can include a AAV capsids or packages. It should be understood that, as used herein, the terms “packages” and capsids are used interchangeably to refer to the structures of the AAV's or other viral capsids. The sample can also include an impurity. Non-limiting examples of impurities include impurities from a nan-electrospray ionization process, solvent ions, and salts.

At step 254, the impurity can be fragmented using the surface induced dissociation device. Optionally, the surface induced dissociation device can be configured so that most or all of the impurity is fragmented without fragmenting the viral capsids. As a non-limiting example, the SID device can be configured to produce an SID voltage of less than 100V to fragment impurities without fragmenting the viral capsids. The surface induced dissociation device can output the fragments of the impurity and the viral capsids.

At step 256, the viral capsids and fragments from the SID device can be input into a charge detection mass spectrometer.

At step 258, a characterization of the plurality of viral capsids can be determined using the charge detection mass spectrometer. The characterization of the viral capsids can be any of the viral capsids characteristics described herein. As non-limiting examples, the characterization can be an intact mass measurement of the viral capsids, or the respective ratios of empty viral capsids, partially-filled viral capsids, and fully-filled viral capsids.

### Example 1

An example embodiment of the present disclosure was studied using adeno-associated viral (AAV) vectors. AAV's can be used for gene delivery systems to combat a variety of human diseases. However, one of die remaining challenges of large-scale AAV use as a therapeutic is quantification of the distribution of fully loaded to capsids to empty/partially filled capsids. Methods such as electron microscopy and analytical centrifugation can typically distinguish between empty and full capsids but determining partially filled capsids remains a challenge using conventional methods. Recently, Charge-Detection Mass Spectrometry (CDMS) has emerged as a powerful diagnostic tool that can be used to determine the ratio of empty, partially, and fully filled AAV's. The example embodiment of the present disclosure combines CDMS with surface induced dissociation (SID) for even more detailed structural characterization.

In the example embodiment of the present disclosure, a Q-Exactive UHMR Hybrid Quadrupole-Orbitrap Mass Spectrometer with a nano-electrospray ionization source and generation 1 SID device was used to acquire native mass spectra for different AAV viral particles. The single-particle charge-detection data can be processed using a prototype of the Selective Temporal Overview of Resonant Ions (STORI) analysis by Thermo Scientific and/or by the single ion CDMS method reported by Heck. A calibration of slope is measured and utilized in data processing downstream. A house-built python-based data processing algorithm can be used to process data prior to deconvolution of the mass spectra using UniDecCD's (version 5.0.2) CDMS data processing software.

Detailed structural characterization of macro molecular complexes can be achieved by combining the fragmentation capabilities of SID with the resolving capabilities of CDMS. The study described herein demonstrate theses effects on a widely used therapeutic that is often difficult to characterize by routine methods.

In the example embodiment, AAV's were found to be stable enough to survive lower levels of SID (up to 100 V), with little to no fragmentation seen. In the example embodiment, SID can be used to remove excessive adduction from salt and/or solvent adducted ions, a common side effect of native electrospray ionization. This can be particularly beneficial in studies where different populations of AAV's were present: non-filled, partially-filled, and fully-filled vectors. The ability to remove excessive adduction was found to improve the ability to resolve and quantify the different populations.

While an intact mass measurement can be useful information for a biomolecular complex, stoichiometric, and structural information can also be learned by fragmentation of the molecule in the gas phase. This procedure is routinely run for smaller protein complexes, but this can be difficult for megadalton size complexes due to resolution and charge assignment constraints and supplying sufficient energy to fragment the molecule. However, SID and CDMS can overcome these limitations for megadalton size complexes, and the study described herein demonstrates that with the AAV capsid. The structure of AAV can be made up of 60 subunits of 3 viral proteins (VPs) VP1, VP2, and VP3, and fragmentation of the empty AAV capsid was found to be possible at higher SID collisional voltages (around 235 V). This experiment resulted in a series of different fragments, each aligning with a different oligomeric state of the viral protein. This included peaks associated with monomer, dimer, trimer, pentamer, and 15mer.

### Example 2

Embodiments of the present disclosure were studied using AAV vector packages. An example AAV vector package includes a 4.7-kb single-stranded DNA (ssDNA) genome, which can be used as a gene delivery system for various human diseases. The example AAV capsids (˜3.8 mega Dalton) contain 60 subunits of 3 distinct viral proteins (VPs) with an approximate ratio of 1:1:10. VP3 is the shortest among VPs and shares the common sequences with VP1 and VP2. Only the overlapping region in VP3 is resolved in the atomic resolution structures of AAVs determined by cryo-electron microscopy (cryo-EM) and X-ray crystallography due to the low abundance of VP1/2 and the disordered nature of the extra region in VP1/2. In the resolved structures of capsids, VP monomers assemble into a T=1 icosahedral capsid shell with 2-, 3-, and 5-fold symmetry axes. There is a depression at the 2-fold symmetry axes contributing to conformational changes in response to pH. The trimers with a 3-fold axis of symmetry protrude from the capsid surface and are responsible for receptor binding and antibody recognition. Five VP monomers form a pentamer with a 5-fold axis of symmetry leaving a channel pore in the center to link the inside virus to the outside environment.

Characterization of AAVs, including the stoichiometry of VPs, and the purity and safety of AAV, can be essential in AAV preparations but limited by the low accuracy and large sample consumption of the conventional analytical techniques. The recent advancement of single-particle charge-detection mass spectrometry (CDMS) allows direct measurement of the charge states and mass-to-charge ratio (m/z) simultaneously, making it possible to characterize large (over 1MDa) and highly heterogenous macromolecules, like AAVs. Previous studies have focused on using CDMS to determine the ratio of empty, partially, and fully filled AAVs. Embodiments of the present disclosure can use surface-induced dissociation (SID) in tandem with CDMS as a powerful tool for more detailed structural characterization of intact AAV capsids.

AAVs were found to be stable enough to survive maximum CID activation, with little to no fragmentation seen. In contrast, SID was found to be a beneficial method that could be useful for dissociating the AAV capsids. The intact mass of empty AAV8 was measured first by CDMS and the major population was 3.72 MDa corresponding to an empty capsid. There is also a minor population (4.14 MDa) with a mass higher than empty AAV8 possibly due to the possible incomplete removal of the genome. The charge envelope of empty AAV8 was isolated and subjected to SID, resulting in a series of different fragments, each aligning with a different oligomeric state of the viral protein.

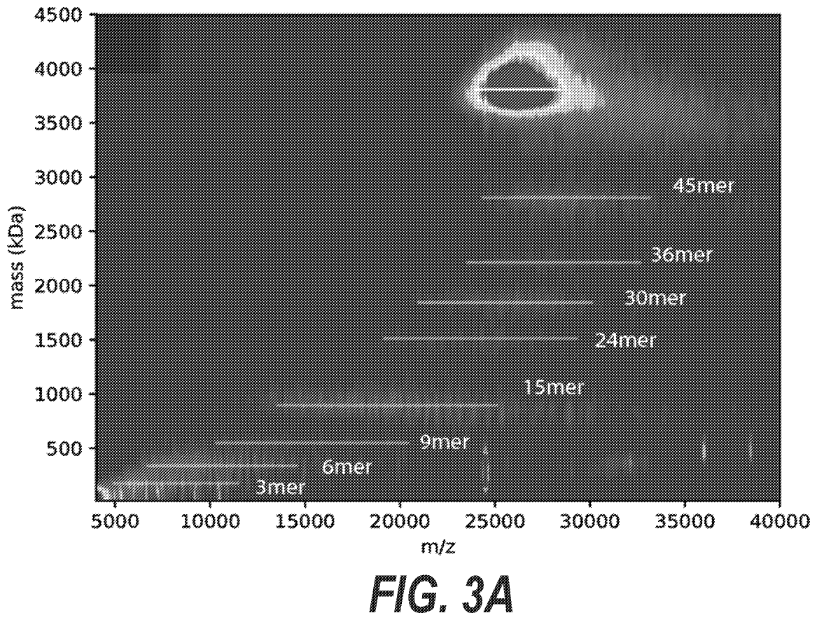

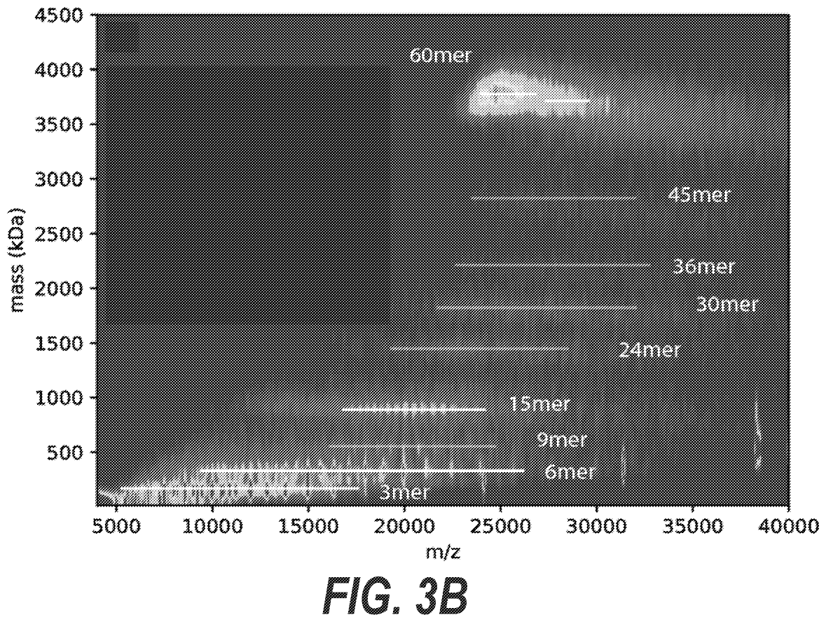

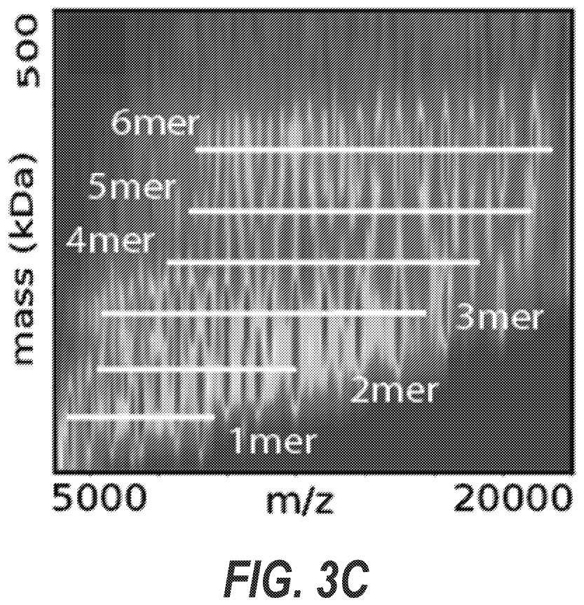

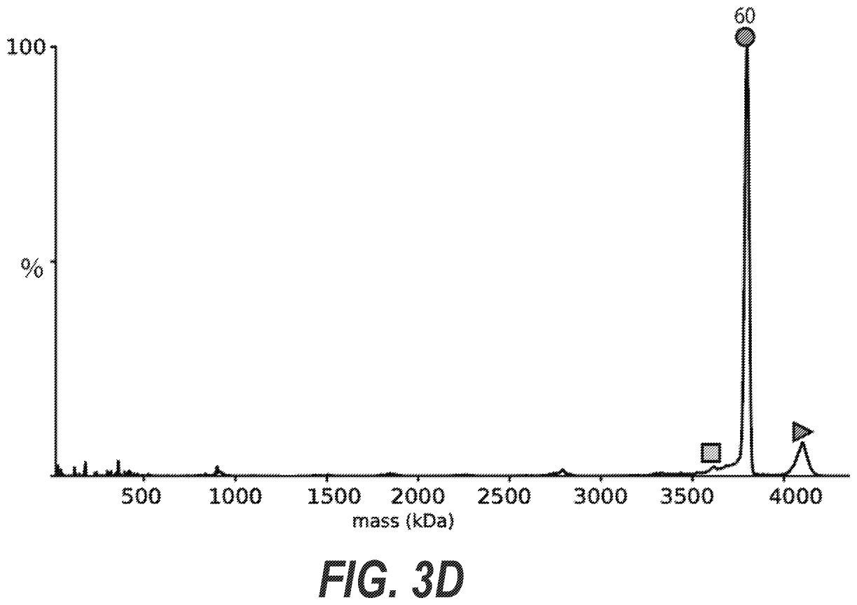

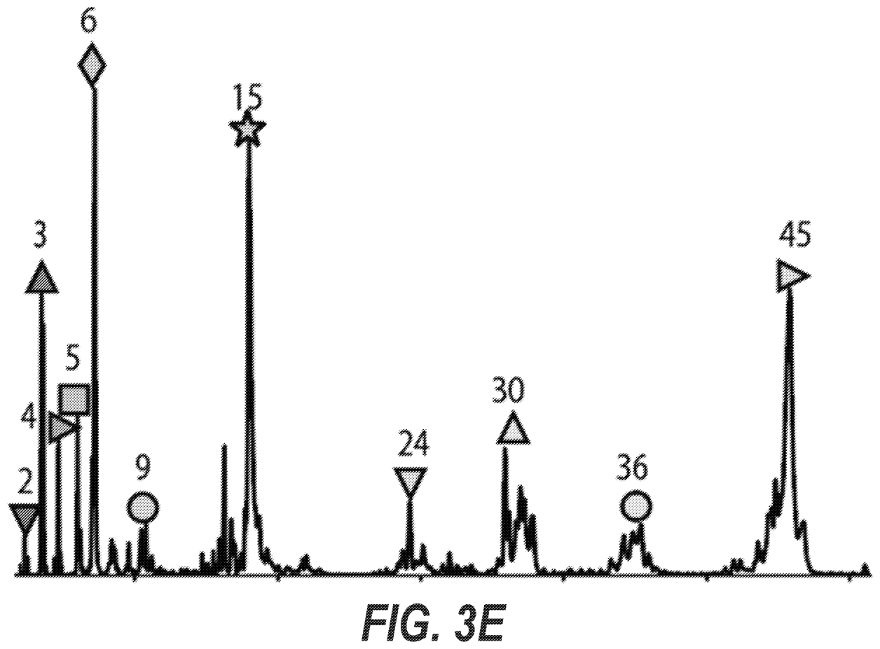

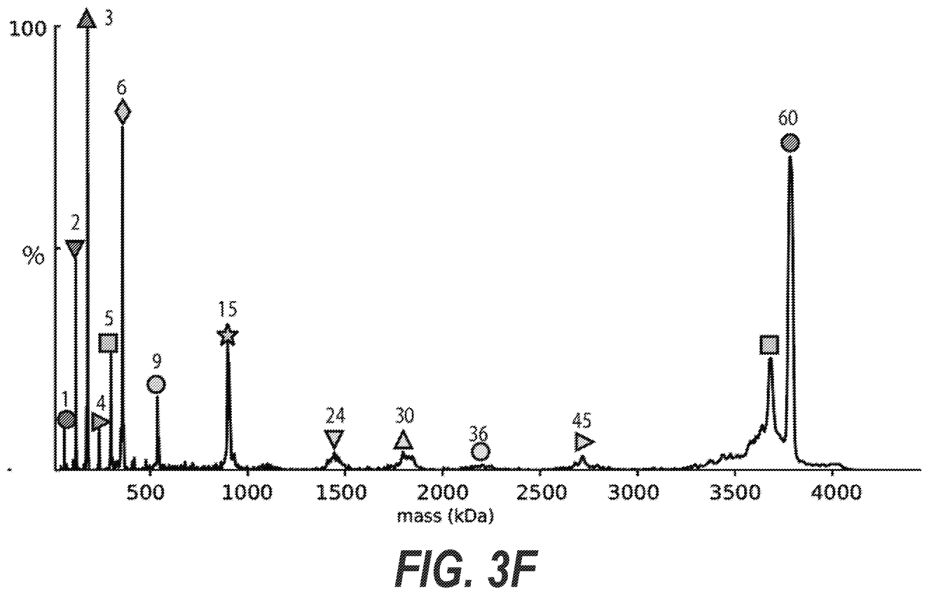

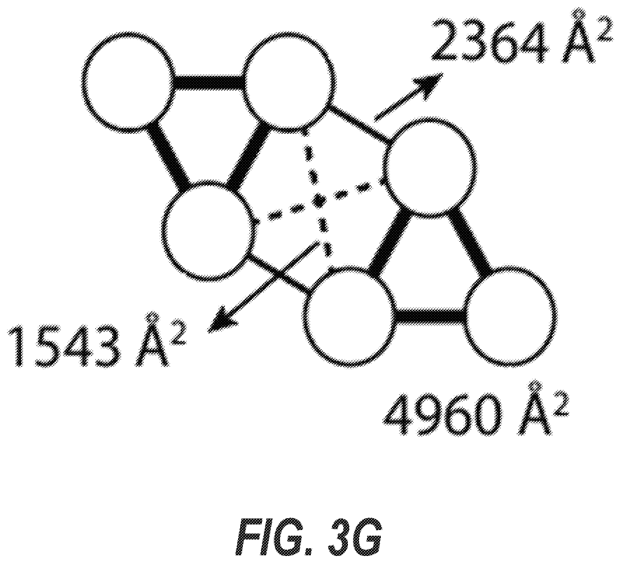

FIGS. 3A-3G illustrate that, at low SID voltage (125V), fragments associated with trimer, hexamer, 9mer, 15mer, 24mer, 30mer, and 45mer were observed FIG. 3A illustrates a mass-m/z plot of an SID spectra from empty AAV8 capsid acquired with an SID voltage of 125V, according to an example embodiment of the present disclosure. FIG. 3B illustrates a mass-m/z plot of an SID spectra from empty AAV8 capsid acquired with an SID voltage of 235V, according to an example embodiment of the present disclosure. FIC. 3C illustrates the low-mass region of FIG. 3B. FIG. 3D illustrates a SID-CDMS spectrum processed and deconvolved by UniDec CD, where the SID voltage was 125V. FIG. 3E illustrates an example spectra from the low-mass region of FIG. 3D. FIG. 3F illustrates a SID-CDMS spectrum processed and deconvolved by UniDec CD, where the SID voltage was 235V. Finally, FIG. 3G illustrates interface areas of AAV8 60mer as determined by PISA analysis (PDB ID: 2QA0). Trimer has a higher interface area, consistent with the trimeric fragments observed in SID. The data shown in FIG. 3A-3G are STORI (Selective Temporal Overview of Resonant Ions) data deconvolved with UniDec.

In the example embodiment described with reference to FIGS. 3A-3G, the fragments are composed of VP trimers, which is consistent with the native topology of AAV that 20 VP trimers assemble into a 60mer capsid. When considering the top 3 interfaces determined by the crystal structure, the total interface areas of VP trimers required to cleave are 14880 Å² which is much higher than those of the interface between trimers (7814 Å²). This shows the tendency to cleave along the ⅖ fold wall primarily when colliding with the surface, releasing 15mer and 45mer. Both 15mer and 45mer can further dissociate at the weaker interfaces, producing additional fragments. Increasing the SID voltage allows higher-intensity monomers, dimers, trimers, tetramers, pentamers, and hexamers to be observed in the low/z region. Those extensive fragments can be due to the secondary dissociation from SID fragments, e.g., monomers and dimers are possible from trimers. Similarly, 9mer is likely to be the complementary fragments of hexamer released from the 15mer with the fact that the relative intensity of 15mer decreases compared to that in low SID. Validation of the secondary dissociation requires the collection of SID energy-resolved mass spectra or the tandem SID in future work. In addition to the verification of AAV topology, SID-CDMS also shows the possibility of the direct examination of VP composition from intact AAVs. The fragments below 15mer contain VP3 only and different combinations of VPs are found in 24mer, 30mer, and 45mer. This is consistent with the fact that the VPs are the most abundant viral proteins with a theoretical percentage of over 83%. The additional sequence in VP2 and VP3 might also provide extra interaction locally, making them harder to be cleaved directly.

One or more of the functions for changing functional settings, such as electrical properties including, but not limited to attraction, repulsion, applying various potentials to one or more portions of the components of SID devices described above in accordance with certain embodiments may be automatic and/or may be computer-controlled, for example by a processing device of a computer executing computer-readable instructions (e.g., a programmable processor coupled to a memory storing computer-readable instructions which, when executed by the processor, cause a computer to perform specific functions) for automating, coordinating and/or otherwise dictating one or more of the above-mentioned settings, changes, and/or other functions. An example computing device 400 is illustrated in FIG. 4.

It should be appreciated that the logical operations described herein with respect to the various figures may be implemented (1) as a sequence of computer-implemented acts or program modules (i.e., software) running on a computing device (e.g., the computing device described in FIG. 4), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special-purpose digital logic, and any combination thereof. It should also be appreciated that more or fewer operations may be performed than shown in the figures and described herein. These operations may also be performed in a different order than those described herein.

Referring to FIG. 4, an example computing device 400 upon which the methods described herein may be implemented is illustrated. It should be understood that the example computing device 400 is only one example of a suitable computing environment upon which the methods described herein may be implemented. Optionally, the computing device 400 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 400 typically includes at least one processing unit 406 and system memory 404. Depending on the exact configuration and type of computing device, system memory 404 may be volatile (such as random access memory (RAM)), non-volatile (such as read-only memory (ROM), flash memory, etc.), or some combination of the two. This most basic configuration is illustrated in FIG. 4 by dashed line 402. The processing unit 406 may be a standard programmable processor that performs arithmetic and logic operations necessary for operation of the computing device 400. The computing device 400 may also include a bus or other communication mechanism for communicating information among various components of the computing device 400.

Computing device 400 may have additional features/functionality. For example, computing device 400 may include additional storage such as removable storage 408 and non-removable storage 410 including, but not limited to, magnetic or optical disks or tapes. Computing device 400 may also contain network connection(s) 416 that allow the device to communicate with other devices. Computing device 400 may also have input device(s) 414 such as a keyboard, mouse, touch screen, etc. Output device(s) 412 such as a display, speakers, printer, etc. may also be included. The additional devices may be connected to the bus in order to facilitate communication of data among the components of the computing device 400. All these devices are well-known in the art and need not be discussed at length here.

The processing unit 406 may be configured to execute program code encoded in tangible, computer-readable media. Tangible, computer-readable media refers to any media that is capable of providing data that causes the computing device 400 (i.e., a machine) to operate in a particular fashion. Various computer-readable media may be utilized to provide instructions to the processing unit 406 for execution. Example tangible, computer-readable media may include, but is not limited to, volatile media, non-volatile media, removable media and non-removable media implemented in any method or technology for storage of information such as computer-readable instructions, data structures, program modules or other data. System memory 404, removable storage 408, and non-removable storage 410 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 406 may execute program code stored in the system memory 404. For example, the bus may carry data to the system memory 404, from which the processing unit 406 receives and executes instructions. The data received by the system memory 404 may optionally be stored on the removable storage 408 or the non-removable storage 410 before or after execution by the processing unit 406.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language and it may be combined with hardware implementations.

The various embodiments described above are provided by way of illustration only and should not be construed to limit the scope of the present disclosure. Those skilled in the art will readily recognize that various modifications and changes may be made to the present disclosure without following the example embodiments and implementations illustrated and described herein, and without departing from the spirit and scope of the disclosure and claims here appended. Therefore, other modifications or embodiments as may be suggested by the teachings herein are particularly reserved.

### LIST OF REFERENCES

[1] Barnes, L. F., Draper, B. E., Chen, Y. T., Powers, T. W. and Jarrold, M. F., 2021. Quantitative analysis of genome packaging in recombinant AAV vectors by charge detection mass spectrometry. Molecular Therapy-Methods & Clinical Development, 23, pp. 87-97.
[2] Barnes, L. F., Draper, B. E. and Jarrold, M. F., 2022. Analysis of thermally driven structural changes, genome release, disassembly, and aggregation of recombinant AAV by CDMS. Molecular Therapy-Methods & Clinical Development, 27, pp. 327-336.
[3] Dobnik D, Kogovsek P, Jakomin T, Kosir N, Tusek Znidaric M, Leskovec M, et al. Accurate quantification and characterization of adeno-associated viral vectors. Front Microbiol. 2019; 10:1570.
[4] Drouin, L. M. and Agbandje-McKenna, M., 2013. Adeno-associated virus structural biology as a tool in vector development. Future virology, 8(12), pp. 1183-1199.
[5] Ebberink, E. H., Ruisinger, A., Nuebel, M., Thomann, M. and Heck, A. J., 2022. Assessing production variability in empty and filled adeno-associated viruses by single molecule mass analyses. Molecular Therapy-Methods & Clinical Development, 27, pp 491-501.
[6] Girod, A., Wobus, C. E., Zádori, Z., Ried, M., Leike, K., Tijssen, P., Kleinschmidt, J. A. and Hallek, M., 2002. The VP1 capsid protein of adeno-associated virus type 2 is carrying a phospholipase A2 domain required for virus infectivity. Journal of General Virology, 83(5), pp. 973-978.
[7] Kafader, J. O., Melani, R. D., Durbin, K. R., Ikwuagwu, B., Early, B. P., Fellers, R. T., Beu, S. C., Zabrouskov, V., Makarov, A. A., Maze, J. T. and Shinholt, D. L., 2020. Multiplexed mass spectrometry of individual ions improves measurement of proteoforms and their complexes. Nature methods, 17(4), pp. 391-394.
[8] Kuck D, Kern A, Kleinschmidt J A. Development of AAV serotype-specific ELISAs using novel monoclonal antibodies. J Virol Methods, 2007; 140:17-24.
[9] Kostelic, M. M., Ryan, J. P., Brown, L. S., Jackson, T. W., Hsieh, C. C, Zak, C. K, Sanders, H. M., Liu, Y., Chen, V. S., Byrne, M. and Aspinwall, C. A., 2022. Stability and dissociation of adeno-associated viral capsids by variable temperature-charge detection-mass spectrometry Analytical Chemistry, 94(34), pp. 11723-11727.
[10] Mietzsch, M., Pénzes, J. J. and Agbandje-McKenna, M., 2019. Twenty-five years of structural parvovirology. Viruses, 11(4), p. 362.
[11] Nam, H. J., Gurda, B. L., McKenna, R., Potter, M., Byrne, B., Salganik, M., Muzyczka, N. and Agbandje-McKenna, M., 2011. Structural studies of adeno-associated virus serotype 8 capsid transitions associated with endosomal trafficking. Journal of virology, 85(22), pp. 11791-11799.
[12] Nam, H. J., Lane, M. D., Padron, E., Gurda, B., McKenna, R., Kohlbrenner, E., Aslanidi, G., Byrne, B., Muzyczka, N., Zolotukhin, S. and Agbandje-McKenna, M., 2007. Structure of adeno-associated virus serotype 8, a gene therapy vector. Journal of virology, 8/(22), pp. 12260-12271.
[13] Pierson, E. E., Keifer, D. Z., Asokan, A. and Jarrold, M. F., 2016. Resolving adeno-associated viral particle diversity with charge detection mass spectrometry. Analytical chemistry, 88(13), pp. 6718-6725.
[14] Sommer J M, Smith P H, Parthasarathy S, Isaacs J, Vijay S, Kieran J, et al. Quantification of adeno-associated virus particles and empty capsids by optical density measurement. Mol Ther. 2003; 7:122-8.
[15]Wörner, T. P., Bennett, A., Habka, S., Snijder, J., Friese, O., Powers, T., Agbandje-McKenna, M. and Heck, A. J., 2021. Adeno-associated virus capsid assembly is divergent and stochastic. Nature communications, 12(1), pp. 1-9.
[16]Wörner, T. P., Snijder, J., Bennett, A, Agbandje-McKenna, M., Makarov, A. A. and Heck, A. J., 2020. Resolving heterogeneous macromolecular assemblies by Orbitrap-based single-particle charge detection mass spectrometry. Nature methods, 17(4), pp 395-398.

1. A system for characterizing the structure of a sample, comprising:
a surface induced dissociation (SID) device configured to receive the sample, the sample comprising viral capsids;
a charge detection mass spectrometer (CDMS) operably coupled to the SID device and configured to receive fragmented viral capsids from the SID device, wherein the CDMS is configured to determine a characterization of the structure of the viral capsids.

2. The system of claim 1, wherein the CDMS comprises a nano-electrospray ionization source.

3. The system of claim 1, wherein the SID device is configured to perform SID on the viral capsids.

4. The system of claim 1, wherein the SID device is configured to perform SID on an impurity in a sample.

5. The system of claim 1, wherein the SID voltage is less than 100 volts.

6. The system of claim 1, wherein the SID voltage is greater than 230 volts.

7. The system of claim 1, wherein the characterization of the structure of the plurality of viral capsids comprises an intact mass measurement of the viral capsids.

8. The system of claim 1, wherein the characterization of the structure of the plurality of viral capsids comprises the respective ratios of empty viral capsids, partially-filled viral capsids, and fully filled viral capsids.

9. A method of characterizing the structure of viral capsids, comprising:
inputting a plurality of viral capsids into an SID device;
fragmenting the plurality of viral capsids using the SID device to create a plurality of viral capsid fragments;
inputting the plurality of viral capsid fragments into a CDMS;
determining a characterization of the plurality of viral capsids using the CDMS; and
outputting a plurality of peaks.

10. The method of claim 9, wherein the CDMS comprises a nano-electrospray ionization source.

11. The method of claim 9, wherein fragmenting the plurality of viral capsids using the SID device comprises configuring the SID device to produce a collision voltage of greater than 230 volts.

12. (canceled)

13. The method of claim 9, wherein the plurality of peaks correspond to monomer, dimer, trimer, pentamer and 9mer, 15mer, 24mer, 30mer, 36, 45mer, and 57mer fragments.

14. The method of claim 9, wherein the characterization of the plurality of viral capsids comprises the respective ratios of a number of empty viral capsids, a number of partially-filled viral capsids, and a number of fully filled viral capsids.

15. A method of characterizing the structure of sample, comprising:
inputting the sample into an SID device, the sample comprising a plurality of viral capsids and an impurity, wherein the SID device is configured to fragment the impurity without fragmenting the viral capsids;
fragmenting the impurity using the SID device;
inputting the plurality of viral capsids into a CDMS; and
determining a characterization of the plurality of viral capsids using the CDMS.

16. The method of claim 15, wherein the sample is prepared using a nano-electrospray ionization source.

17. The method of claim 15, wherein the impurity comprises a salt or a solvent ion.

18. (canceled)

19. The method of claim 15, wherein the impurity comprises a byproduct of an electrospray process.

20. The method of claim 15, wherein the SID device is configured to produce an SID voltage less than 100V.

21. The system of claim 15, wherein the characterization of the plurality of viral capsids comprises an intact mass measurement of the viral capsids.

22. The method of claim 15, wherein the characterization of the plurality of viral capsids comprises the respective ratios of empty viral capsids, partially-filled viral capsids, and fully filled viral capsids.
